(19) United States
(12) Patent Application Publication
(10) Pub. No.: US 2024/0263380 A1
(43) Pub. Date: Aug. 8, 2024

(54) LAUNDRY TREATMENT CABINET WITH A DEVICE FOR TREATING THE INTERIOR AIR

(71) Applicant: Miele & Cie. KG, Gütersloh (DE)

(72) Inventors: Felix SCHÄFER, Bielefeld (DE); Patrick SCHIMKE, Gütersloh (DE)

(21) Appl. No.: 18/554,203

(22) PCT Filed: Apr. 5, 2022
(86) PCT No.: PCT/EP2022/059006

§ 371 Date: Oct. 6, 2023

(30) Foreign Application Priority Data
Apr. 9, 2021 (BE) 2021/5276

(51) Int. Cl. D06F 58/10; A61L 9/20; B01D 53/00; B01D 53/30; B01D 53/86; B01D 53/88; B01J 21/06; B01J 35/39; D06F 34/26; D06F 58/20; D06F 105/16; D06F 105/30; D06F 105/32

(52) U.S. Cl. CPC D06F 58/10; A61L 9/205; B01D 53/007; B01D 53/30; B01D 53/8687; B01D 53/8696; B01D 53/885; B01J 21/063; B01J 35/39; D06F 34/26; D06F 58/20; A61L 2209/11; A61L 2209/12; B01D 2255/20707; B01D 2255/802; B01D 2257/708; B01D 2257/91; B01D 2259/804; D06F 2105/16; D06F 2105/30; D06F 2105/32

(57) ABSTRACT

Disclosed herein is a laundry treatment cabinet that includes a device for treating the interior air. The disclosed laundry treatment cabinet includes a housing; at least one interior space for receiving laundry with an access opening that can be closed by means of a door; an assembly for generating or conveying the process air through the interior space; a device for treating the interior air, wherein the device comprises a reactor chamber with a catalyst means attached therein; and a control apparatus for activating and deactivating the assembly for generating the process air. The laundry treatment cabinet also includes a flow generator for conveying the air through the reactor chamber.

The invention relates to a laundry treatment cabinet, comprising
a housing;
at least one interior space for receiving laundry with an access opening that can be closed by means of a door,
an assembly for generating or conveying the process air through the interior space;
a device for treating the interior air, wherein the device comprises a reactor chamber with a catalyst means attached therein;
a control apparatus for activating and deactivating the assembly for generating the process air.

EP 2267213 A1 discloses a laundry treatment cabinet with a device for treating the process air, wherein the device comprises a treatment space and a flow generator for conveying the air through the treatment space, in which the air is treated photocatalytically. This treatment is carried out continuously, i.e., uninterrupted during operation of the process air blower, by means of an excitation light.

The object of the invention is therefore to provide a laundry treatment cabinet which provides an improved and need-based treatment of the laundry in a simple manner.

According to the invention, at least this object is solved by a storage cabinet having the features of claim 1. Advantageous features and further embodiments of the invention can be found in the respective dependent claims.

The advantages that can be achieved with the invention are that the process air is conditioned in a simple manner as needed. In particular, due to the treatment cabinet proposed here, odour emissions can be reduced in a targeted manner or at predetermined times depending on the requirements predetermined by the user. For this purpose, the device for air treatment comprises its own flow generator for conveying the air through the reactor chamber, in addition to the assembly or the flow generator in the assembly for providing the process air. The interior space represents the treatment space for the laundry and is not movable relative to the housing or assembly. The laundry is suspended in the interior space, preferably by means of clothes hangers, wherein means can be present to move the clothes hangers in order to improve the flow of the process air through the clothing. Furthermore, a steam generator can be provided in the cabinet in order to introduce steam into the interior space, which can in turn act on the laundry. The movement of the clothes hangers and the activity of the steam generator are preferably controlled by the control apparatus.

In an expedient embodiment, the assembly is arranged in the base region of the cabinet, below the interior space, wherein the device for air treatment is arranged in the upper region of the cabinet, above the interior space. This means that the process air is admitted into the interior space from below and extracted, wherein a circulation of the process air is provided within the interior space, which flows through the entire interior space or reaches at least almost the entire interior space. The process air thus reaches and correspondingly dries or conditions the laundry located in the interior space, for example with steam or a treatment agent.

In an expedient embodiment, the air to be treated is extracted through an inlet in the upper region of the interior space and admitted into the reactor chamber. After the treatment, the air is admitted into the interior space again from the reactor chamber through the air outlet in the upper region of the interior space. The air outlet is located at a distance from the air inlet, preferably 10 cm to 50 cm. The air inlet is preferably arranged in the rear region and the air outlet is preferably arranged in the front region or in the central region on the upper side of the interior space. It is thus ensured that a circulation of the air to be treated through the interior space takes place and the process air is achieved at least in a partial region. Overall, all position and direction information relates to the operational installation position of the laundry treatment cabinet.

In an overall expedient embodiment, the control apparatus is further configured to activate and deactivate the device for air treatment, irrespective of the control of the assembly. For this purpose, the control apparatus can be designed and programmed to activate and deactivate the device for treating the interior air on the basis of a predetermined time sequence. This means that the activation times for the conditioning module are specified on the basis of a predetermined schedule so that the module is active only at certain times. This saves energy because the device is always inactive when no conditioning or disinfection of the process air is necessary. The control apparatus is preferably configured to activate and deactivate the flow generator of the device.

In an overall expedient embodiment, the storage cabinet comprises an operating device which is configured to provide a user input for the progression of the activity of the device and to transmit said user input to the control apparatus. Thus, for example, a schedule of when the device is to be activated for air treatment can be specified by the user. Furthermore, the intensity of the air treatment can be set. Furthermore, the intensity and/or the duration of the air treatment can be assigned to a particular laundry type. Then, the user only needs to enter the laundry type to be treated by means of the operating device, wherein the intensity and/or duration of the air treatment is set according to the assignment to the initialized laundry type.

In an overall advantageous embodiment, the device for air treatment comprises the following:
a reactor chamber;
at least one activatable catalyst mat arranged in the reactor chamber, which can be activated by means of irradiation, preferably UV radiation;
a blower for conveying the air from the interior space through the reactor chamber, so that the air to be treated flows through or flows along the side of the catalyst mat;
radiation means for activating said catalyst mat, wherein the blower and the radiation means can be switched on and off by means of the control apparatus.

To set the intensity of the catalyst effect and overall air purification, the control apparatus is expediently programmed and configured such that the pulse/pause ratio between the switch-on time and the switch-off time determines the intensity of the air purification. This embodiment is particularly easy to implement because time progressions that include only switching on and off of actuators can be programmed and complex hardware changes are avoided.

In another embodiment, the control apparatus can be programmed and configured such that the radiation means can be activated continuously with different powers and the control apparatus is configured to switch the corresponding power to the radiation means. This requires a possibility of, for example, a configurable amplifier or circuit breaker, which correspondingly receives a corresponding power value from the control apparatus.

In an advantageous development of the laundry treatment cabinet, the device comprises a sensor for detecting the air quality, preferably a VOC sensor (volatile organic compounds), with which odours can be detected. The sensor is connected to the control apparatus so that it can set the duration of the treatment and/or the intensity of the treatment, so that a good result is always achieved in the shortest time. In this case, the sensor is attached in a protected manner behind the inlet, optionally behind the inlet filter within the reactor chamber.

In an overall expedient embodiment, the radiation agent comprises at least one UV LED, wherein the at least one activatable catalyst mat comprises titanium dioxide as a catalyst material or consists of titanium dioxide. The catalyst mat preferably comprises a carrier layer, for example a woven fabric, gauze, non-woven fabric or an open-pore foam, which is coated on one or both sides with the catalyst material. This ensures durability and catalytic effect throughout the service life of the laundry treatment cabinet.

In an overall advantageous embodiment, multiple catalyst mats are attached at a distance from one another in the reactor chamber. A large reactive area for the catalyst material is thereby provided with compact dimensions of the reactor space.

In an advantageous embodiment or development, the catalyst mats are attached in the reactor chamber longitudinally in the direction of flow, so that the air to be treated can flow through the intermediate spaces between the spaced-apart catalyst mats and through the intermediate space between the catalyst mat and the adjacent chamber wall of the reactor chamber in each case.

In an advantageous embodiment, the mat or mats are designed to be pleated, whereby the active or activatable surface of each mat is increased.

In an overall advantageous embodiment, the reactor chamber is arc-shaped, semicircular or dome-shaped, wherein the apex is located at the top. The catalyst mats are shaped in an arc-shaped, semicircular or dome-shaped manner according to the shape of the reactor chamber. As a result, a single radiation source can be arranged at the centre point or focal point, which radiation source fans out the activating light and thereby scatters it broadly and reaches the entire surface of said stretched mat with almost uniformly distributed intensity.

In an overall expedient embodiment, the radiation means is designed such that it can be activated with different powers, wherein the control apparatus is configured to switch the corresponding power to the radiation means.

In a further, overall expedient embodiment, the control apparatus is configured to activate the blower and the radiation means for period 10% to 80%, based on the entire treatment time for the laundry. This protects the air treatment device and ensures a long service life because no process air is conveyed through the reactor chamber when the air treatment device is inactive.

In an overall advantageous further development, the control apparatus is configured to activate the blower and the radiation means depending on the laundry type to be treated or the type of care, preferably with respect to the duration and/or intensity.

An embodiment of the invention is shown purely schematically in the drawings and is described in more detail below. In the drawings:

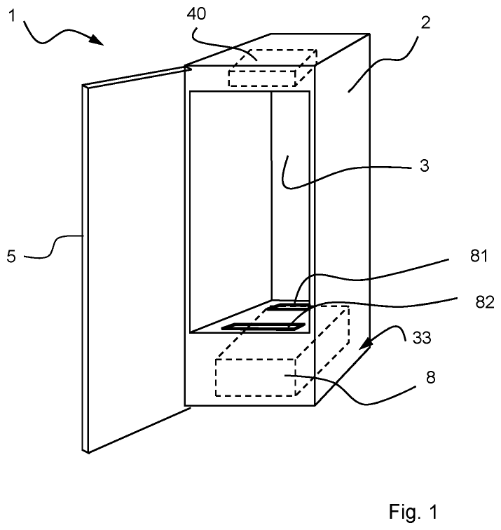

FIG. 1, 2: show a laundry treatment cabinet with a view into the interior space in different views;

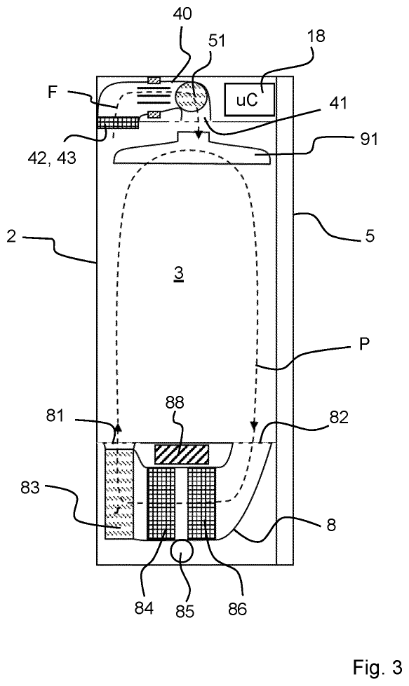

FIG. 3: shows an exemplary outlined, sectional side view of the laundry treatment cabinet;

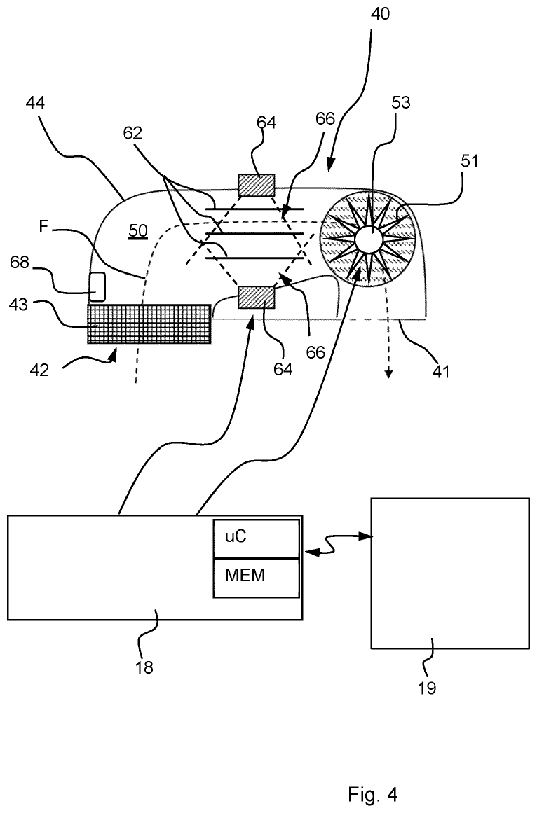

FIG. 4, 7: schematically show a device for air treatment in various embodiments; and

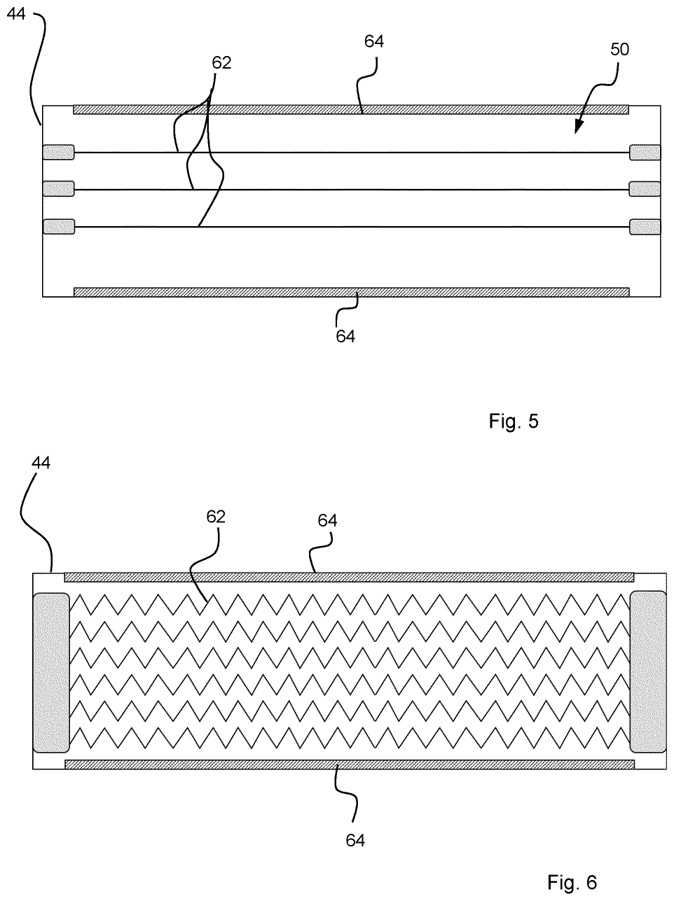

FIG. 5, 6: show the catalyst mat of the device in various embodiments.

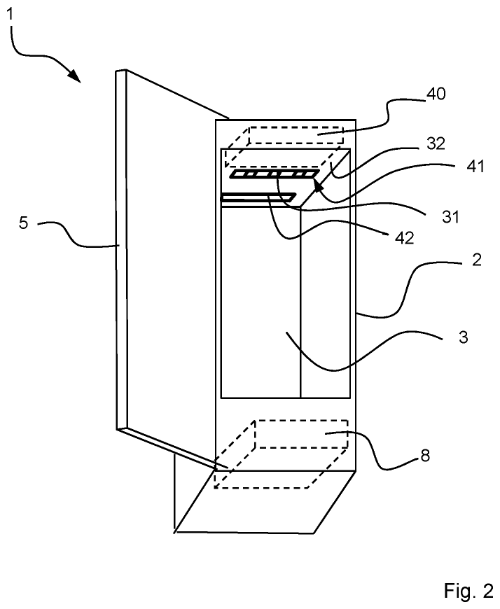

FIG. 1 shows a laundry treatment cabinet 1 with an open door 5 in the viewing direction obliquely from above, in which the interior space 3 is visible. In this example, the laundry treatment cabinet 1 is a drying cabinet comprising a housing 2, in the interior space 3 of which means 31 (FIG. 2) for suspending laundry or clothes hangers 91 are located. In the interior of the housing 2, the laundry treatment cabinet 1 comprises a heat pump assembly 8 for providing and conveying the process air, which is admitted through the inlet opening 81 into the interior space 3 and is conveyed through the outlet opening 82 out of the interior 20) space 3 to the assembly 8. In this example, the assembly 8 is arranged in the lower region 33 of the housing 2. In this illustration, the front side of the door 5 is designed to be opaque, but it can be transparent so that it is possible to see inside in the closed position. The laundry treatment cabinet 1 further comprises a device 40 for treating the air in the interior space 3 and a control apparatus 18, which preferably comprises a microcontroller uC with an associated memory MEM in order to control, or activate and deactivate, the cooling assembly 8 and the device 40 for air treatment according to the program stored in the memory MEM (FIG. 4).

FIG. 2 shows the laundry treatment cabinet 1 with the door 5 open in the viewing direction obliquely from below, in which the interior space 3 is visible. In this view, the device 40 for treating the air can be seen in the upper region of the housing 2 above the interior space 3. The air inlet 42 for the device 40 is located at the rear region on the upper side 32 of the interior space 3, through which the air F to be treated is extracted from the interior space 3. The air outlet 41 is located in the central region on the upper side 32 of the interior space 3, through which the treated air F is blown out of the device 40 into the interior space 3.

FIG. 3 shows the laundry treatment cabinet 1 in a schematic sectional illustration with door 5 closed during operation. The heat pump assembly 8 for generating the process air P is located in the base region of the housing 2. The assembly 8 comprises a heat pump circuit with a cooling heat exchanger 86 (evaporator), a heating heat exchanger 84 (condenser) and a compressor 85 in order to convey the refrigerant contained in the heat pump circuit through the individual components. By means of the process air blower 83, the air flow of the process air P is generated, which passes through the outlet 82 to reach the cooling heat exchanger 86 and is dehumidified there. Subsequently, the dry, cool air reaches the heating heat exchanger 84, which it leaves again after being warmed. Further downstream is the process air blower 83, which conveys the now dried and heated process air into the interior space 3 through the inlet 81. It can also be seen that the inlet 81 is arranged close to the rear wall of the interior space 3. The outlet 82 is arranged at a distance from the inlet and close to the door 5. Thus, an air flow P that reaches the entire interior space 3 is generated.

FIG. 3 also shows by way of example that the laundry treatment cabinet 1 comprises a steam generator 88. This can also be activated and deactivated by means of the control apparatus 18 in order to supply steam to the interior space 3 or the process air P.

The device 40 for air treatment is arranged in the upper region of the housing 2 above the interior space 3. It comprises
a housing 52;
a reactor chamber 50 located therein;
at least one activatable catalyst mat 62 that is arranged in the reactor chamber 50 and can be activated by means of irradiation, preferably UV radiation;
a blower 51 for conveying the air F out of the interior space 3 through the reactor chamber 50;
radiation means 64 (FIG. 4) for activating said catalyst mat 62.
   The fan 51 and the radiation means 64 are switched on and off by means of the control apparatus 18, as a function of programmed specifications or those set by the user. The air F to be treated passes through the inlet 42 to reach the reactor chamber 50, wherein the treated air F is blown through the outlet 41 out of the reactor chamber into the interior space 3.

The air is thereby disinfected, odours removed and germs or other unwanted spores are killed. A filter 43 on the inlet side 42 serves to prevent dust and lint from entering the reactor chamber 50. The filter 43 can optionally be used and comprises a non-woven material, activated charcoal or woven material, preferably a combination of the components mentioned.

In the embodiment shown in FIG. 4, the device 40 for air treatment is shown in a sketched detailed illustration. Within the housing 44, a channel 50 is formed as the reactor chamber, which extends from the air inlet 42 to the air outlet 41. The catalyst mats 62 are arranged within the channel 50. The blower 51 is driven by means of the blower motor 53 and serves to generate an air flow F and thereby extract the air F into the channel 50 through the inlet opening 42 and to guide it along the catalyst mats 62 and to convey it along or through the catalyst mats 62 and out of the air outlet 41 back out of the channel 50. The discharged cleaned air F is then conveyed back into the interior space 3 (FIG. 3) of the storage cabinet 1. Furthermore, radiation means 64 are attached in the channel 50, or reactor space, and are placed in such a way that they can irradiate the activatable filter layer of the mats 62 when they are switched on or activated by the control apparatus 18. In this case, the beams 66 are conically fanned out so that they reach an, if possible the entire, area of the activatable mats 62. Preferably, the filter layer comprises a material which brings about ionization of the contaminated air F by means of UV radiation and thereby converts, for example, odour-forming molecules accordingly. The mat 62 acts as a catalyst here. In the outlined example, two radiation means 64, preferably UV LEDs, are used, so that both light cones 66 together irradiate the entire area of the mats 62 with almost uniform intensity. By means of the UV radiation forms, the catalyst material located in the mat 62 is activated by forming radicals that oxidatively degrade odour-forming molecules. The control apparatus 18 with the microcontroller uC and the associated memory MEM for programs and parameters controls the power of the blower 51, or of the blower motor 53, and the radiation means 64. Furthermore, the control apparatus 18 is connected to the operating device 19 so that user inputs with regard to the intensity of the air purification can reach the control apparatus 18 as parameters. The blower 51 is designed as an axial fan in the example outlined; other types of fans such as radial fans can also be used if the housing 44 with the channel 50 formed therefrom is designed accordingly. Optionally, the device can be with a sensor 68 for detecting air quality, a VOC sensor (volatile organic compounds), with which odours can be detected. The sensor is connected to the control apparatus 18 so that the latter can set the duration of the treatment and/or the intensity of the treatment as a function of the detected air quality, so that a good result is always achieved in the shortest time. The sensor 68 is attached in a protected manner behind the inlet filter 43 within the reactor chamber 50.

FIG. 5 schematically shows the channel 50 in an enlarged sectional illustration. The radiation means 64 are flat and attached to the inside of the housing 44 so that an at least almost uniform irradiation of the mats 62 is ensured. The mats 62 are designed as flat plates. The mats 62 can preferably be designed to be partially transparent so that the inner mats 62 are also reached by the activation light 66.

FIG. 6 schematically shows the channel 50 in an enlarged sectional illustration. The radiation means 64 are flat and attached to the inside of the housing 44 so that an at least almost uniform irradiation of the mats 62 is ensured. In this embodiment, the mats 62 are designed to be pleated, as a result of which the surface of the individual mats 62, along or at least partially through which the air F to be treated can flow, is enlarged. The mats 62 can preferably be designed to be partially transparent so that the inner mats 62 are also reached by the activation light 66.

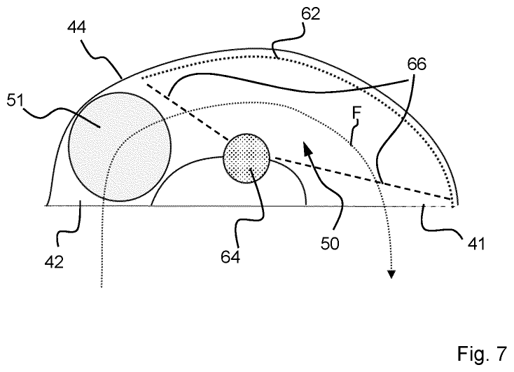

FIG. 7 schematically shows the channel 50 in an enlarged lateral view in a further embodiment. The channel, or reactor chamber 50, is arc-shaped or hemispherical, wherein the catalyst mat 62 is shaped in an arc shape according to the shape of the reactor chamber 50. The radiation means 64 is arranged centrally at the focal point of the arc and is designed to emit the light 66 in a fan shape in the direction of the arc-shaped mat 62. In this example, only one catalyst mat 62 is located In the reactor chamber 50.

1. A laundry treatment cabinet comprising:
a housing;
at least one interior space for receiving laundry with an access opening that is closable by means of a door;
an assembly to produce or convey process air through the interior space;
a device to treat the interior air, wherein the device comprises a reactor chamber with a catalyst means attached therein;
a control apparatus to activate and deactivate the assembly to generate the process air; and
a flow generator to convey the air through the reactor chamber.

2. The laundry treatment cabinet according to claim 1,
wherein the assembly is arranged in the base region of the cabinet, below the interior space, and the device to treat the interior air is arranged in the upper region of the cabinet, above the interior space, wherein the interior space comprises an inlet in the upper region or on the upper side for extracting the air to be treated, and an air outlet in the upper region of the interior space for discharging the treated air into the interior space.

3. The laundry treatment cabinet according to claim 2,
wherein the air inlet is located at a distance from the air outlet, wherein the air outlet is arranged in the front region or in the central region on the upper side of the interior space.

4. The laundry treatment cabinet according to claim 1,
wherein the control apparatus is further configured to activate and deactivate the device to treat the air independently of the controller of the assembly.

5. The laundry treatment cabinet according to claim 4,
wherein the control apparatus is configured to activate and deactivate the flow generator of the device independently of the activation of the assembly to provide the process air.

6. The laundry treatment cabinet according to claim 1,
further comprising an operating device configured to provide user input to activate the device and to transmit said user input to the control apparatus.

7. The laundry treatment cabinet according to claim 1,
wherein the device for air treatment comprises the following:
a reactor chamber;
at least one activatable catalyst mat arranged in the reactor chamber, wherein the at least one activatable catalyst mat is activatable by means of irradiation;
a blower to convey the air out of the interior space through the reactor chamber;
a radiation means to activate the at least one activatable catalyst mat,
wherein the blower and the radiation means is configured to be switched on and off or controlled by the control apparatus.

8. The laundry treatment cabinet according to claim 7, the laundry treatment cabinet further comprising
a sensor to detect the air quality connected to the control apparatus.

9. The laundry treatment cabinet according to claim 7, wherein the radiation means comprises at least one ultraviolet light-emitting diode (UV LED) and the at least one activatable catalyst mat comprises titanium dioxide or titanium dioxide as a catalyst material.

10. The laundry treatment cabinet according to claim 7,
wherein the at least one activatable catalyst mat comprises a support layer comprising a woven fabric, a gauze, a non-woven fabric, or an open-pore foam, wherein the support layer is coated on one or both sides with the catalyst material.

11. The laundry treatment cabinet according to claim 7,
wherein multiple catalyst mats are attached at a distance to each other in the reactor chamber.

12. The laundry treatment cabinet according to claim 11,
wherein the multiple catalyst mats are attached in the reactor chamber longitudinally in the flow direction such that the air to be treated is able to flow through the intermediate spaces between the spaced-apart catalyst mats.

13. The laundry treatment cabinet according to claim 7,
wherein the at least one activatable catalyst mat is pleated.

14. The laundry treatment cabinet according to claim 7,
wherein the reactor chamber is arc-shaped, semicircular, or dome-shaped, and the at least one activatable catalyst mat is shaped in an arc-shaped, semicircular, or dome-shaped manner according to the shape of the reactor space.

15. The laundry treatment cabinet according to claim 1,
wherein the radiation means is activatable with different powers and the control apparatus is configured to switch the corresponding power to the radiation means.

16. The laundry treatment cabinet according to claim 1,
wherein the control apparatus is configured to activate the blower and the radiation means for a time period of between 10% to 80%, based on an entire treatment time for the laundry.

17. The laundry treatment cabinet according to claim 15,
wherein the control apparatus is configured to activate the blower and radiation means depending on a laundry type to be treated.

18. The laundry treatment cabinet according to claim 15, where in the control apparatus is configured to activate the blower and radiation means depending on a duration and/or intensity of treatment.
